(19) United States
(12) Patent Application Publication
(10) Pub. No.: US 2017/0252003 A1
(43) Pub. Date: Sep. 7, 2017

(54) Tissue characterization apparatus

(71) Applicants: Igor Yukov, Orange, CT (US); David Ross, Orange, CT (US)

(72) Inventors: Igor Yukov, Orange, CT (US); David Ross, Orange, CT (US)

(21) Appl. No.: 14/998,914

(22) Filed: Mar. 4, 2016

(51) Int. Cl. A61B 8/00; A61B 8/08; A61B 8/13; A61B 90/00

(52) U.S. Cl. CPC A61B 8/4416; A61B 90/37; A61B 8/08; A61B 8/13; A61B 8/48; A61B 8/5269; A61B 8/463; A61B 8/4483; A61B 8/54; A61B 2090/378; A61B 2560/0493

(57) ABSTRACT

An apparatus for non-invasively determining a type of tissue matter and its state within a living entity consisting of application of two different diagnostic images simultaniously through the same B-scan transducer is disclosed. The apparatus uses a B-scan image visualization as a guiding image information for two-frequency attenuation method to determine an attenuation data for any chosen spot of interest and includes the steps of applying arbitrary waveform generator to produce a B-scan image of the tissue matter to be analyzed, selecting a region of interest on the image, positioning the indicator of the direction of the receiving reflected signals to the spot of interest, detecting reflected signals from said spot of interest, analyzing the reflected signals to determine attenuation data for the tissue matter. The same advanced combined diagnostic examination can be achieved by using any existing on the market ultrasound diagnostic apparatus with a B-scan imaging system by applying interface switch-adapter consisting of switching of some piezoelements of the same B-scan transducer to the two-frequency attenuation apparatus for the period of measurement of the attenuation data to determine a type of tissue matter and its state within a living entity.

### BACKGROUND OF THE INVENTION

The present invention relates to an ultrasound apparatus for non-invasively determining the type of tissue matter or the state of tissue matter in a living body.

For many years specialists in ultrasound diagnostic field trying to develop an ultrasound diagnostic method and apparatus which can give them an information to differentiate type of tissue through measuring attenuation coefficient in a living body or by finding a pattern of the tissue images. There are many attempts to rich this goal by using spectrum analyzes of reflected echo-signals like U.S. Pat. No. 6,007,489 to Yost et al., European Patent No. 11840135 to Hironaka and many more others which could not let them to come up with objective and reliable method for clinical application. Some specialists like European Pat. No. PCT/IB2014/067105 to Schneider, European Pat. No. PCT/CA2014/2014/050480 to Sadeghi, U.S. patent Ser. No. 14/096,960 to Anuja, European Pat. No. PCT/US2014/011631 to Chen and others tried to find a pattern in the tissue images to differentiate the type of tissue. The problem is the reflected signals depend not only on attenuation information from inside of the tissue structure but also on the angle of incident of the ultrasound pulses to reflected surface, its geometry and roughness.

Attempts to find a system employing ultrasound methods for determination the nature of tissue within a living body is still continuing. One such system is disclosed in U.S. Pat. No. 5,361,767 to Yukov. This system determines a type of tissue or the state of the tissue by using developed by the author “Two-Frequency Method” and based on application of the B-scan imaging technique to generate an image of the tissue matter to be examined, selects a region of interest to be analyzed, suggests positioning a device for transmitting an ultrasound signals at least on two different desired frequencies in a desired location relative to the tissue matter to be analyzed and analyzing the echo signals to determine attenuation coefficient for said tissue matter. In the Chines Patent No. CN1113631C to Korotkoff discloses a two-frequency method and apparatus which based on developed “Two-Frequency Method” described in U.S. Pat. No. 5,361,767. Author suggesting an automatically subtraction of reflected echo-signals on two different frequencies and putting the results as two-dimensional attenuation image on the screen. In the U.S. Pat. No. 5,361,767 there is a claim for two-dimensional attenuation image apparatus by using two-frequency method and because of there is no direct dependency between reflected signals and attenuation information as mentioned above the author puts a special requirements to get an objective results. That is why the Patent No. CN1113631C for automatic two-dimensional attenuation image apparatus can not obtain objective attenuation information and it will be impossible to apply it in the clinical environment. U.S. Pat. No. 5,361,767 suggesting to apply a B-scan (two-dimensional) structural image information to find a spot of interest for attenuation measurement and by using the same transducer to transmit an ultrasound signals on two different frequencies to chosen spot of interest to measure the attenuation coefficient through A-mode echo-signals information. Author also suggests to use simultaneous, in sequence or alternative the same transducer for B-scan image visualization and for Two-Frequency Method image to calculate attenuation information. The patent has a very important ommitions by not describing how to implement all these ideas which makes this invention incomplete and in many cases impossible to use in a clinical environment.

In the modern time practically all diagnostic clinical facilities are using ultrasound diagnostic machines with the B-scan image and when examiners see on the screen unusual tissue structure image they try to determine what kind of abnormalities there is and in many cases it is impossible to do that with an existing B-scan imaging information. That is why it is very important to make it possible to add to existing ultrasound diagnostic machines with B-scan image the information of the two-frequency attenuation method and to make it able to the specialists to use these two different diagnostic methods together to differentiate non-invasively the type of the tissue matter or the state of the matter being under examination.

### SUMMERY OF THE INVENTION

It is an object of the present invention to provide a combined diagnostic apparatus which is able to let examiners non-invasively determine the type of the tissue matter and the state of the tissue matter in a living body by applying one transducer simultaniously for generating a B-scan diagnostic image and a two-frequency attenuation image to overcome the aforementioned problems.

It is a further object of the present invention to apply together the existing on the market any B-scan imaging apparatus and the two-frequency attenuation apparatus by using their transducers as one combined transducer for both images to give to the medical specialists possibilities to accurately determine non-invasively a type of tissue matter and the state of the tissue matter being under examination.

It is still a further object of the present invention to provide a reliable switch-adapter to any existing on the market B-scan imaging apparatus to switch some part of the piezoelements inside of B-scan transducer to two-frequency attenuation apparatus for the period of two-frequency examination time to accurately determine non-invasively a type of tissue matter and the state of the tissue matter being under examination.

These and other objects and advantages will become more apparent from the following description and drawings wherein like reference numerals depict like elements.

In accordance with present invention, the apparatus for non-invasively determination a type of tissue and the state of a tissue broadly comprises the steps of:

to apply approximately the same excitation pulses on two different frequencies to a B-scan imaging transducer and also for two-frequency attenuation image simultaniously and by using an imaging technique to generate in a sequence a two B-scan images on two different frequencies of the tissue matter being under examination; selecting a region of interest by pushing a buttons for desired spot of interest or applying voice recognition device to choose that spot of interest of said tissue matter to be analyzed; registering an A-mode echo signals from that chosen spot of interest with an objective attenuation information on two different frequencies; analyzing the A-mode echo signals to determine at least one attenuation data for said tissue matter; or/and a) create a two-dimensional two-frequency overlay colored attenuation image of the spot of interest; or/and b) to show on the screen the attenuation information as a numerical data of that spot of interest;

to apply a two-frequency A-mode transducer separately from B-scan image transducer but as attachment to it; or

to apply a two-frequency A-mode transducer separately from B-scan image transducer but as inserted permanently in the B-scan transducer; or

to apply the same B-scan transducer in sequence or alternate for both methods—for two-dimensional image and for two-frequency attenuation method—by switching certain piezo element(s) inside of that B-scan transducer for the period of the two-frequency method application;

Details of the present invention are set forth in the following detailed description and the accompanying drawings wherein like reference numerals depict like elements.

### BRIEF DESCRIPTION OF THE DRAWINGS

FIG. 1 is a schematic representation of an apparatus which can perform B-scan images on applied two different frequencies in a sequence and at the same time to generate simultaneously with the same excitation pulses a two-frequency method image to determine the attenuation information of the tissue being under examination with choice of numerical attenuation data visualization or/and two-dimensional overlay colored attenuation image of that spot of interest.

FIG. 2 is a schematic representation of the interface switch-adapter for any existing on the market B-scan transducers to switch some of its piezo-element(s) from B-scan image mode to Two-Frequency attenuation image mode;

### DETAILED DESCRIPTION

Existing on the market B-scan image apparatus based on a multi-element transducers and by connecting them to scanning system they produce a two-dimensional (B-scan) image of the organ or tissue being under examination. The Two-Frequency Attenuation method requires a single element transducer only and produces an A-mode (Amplitude Modulated) image from the spot of interest of the organ or tissue being under examination.

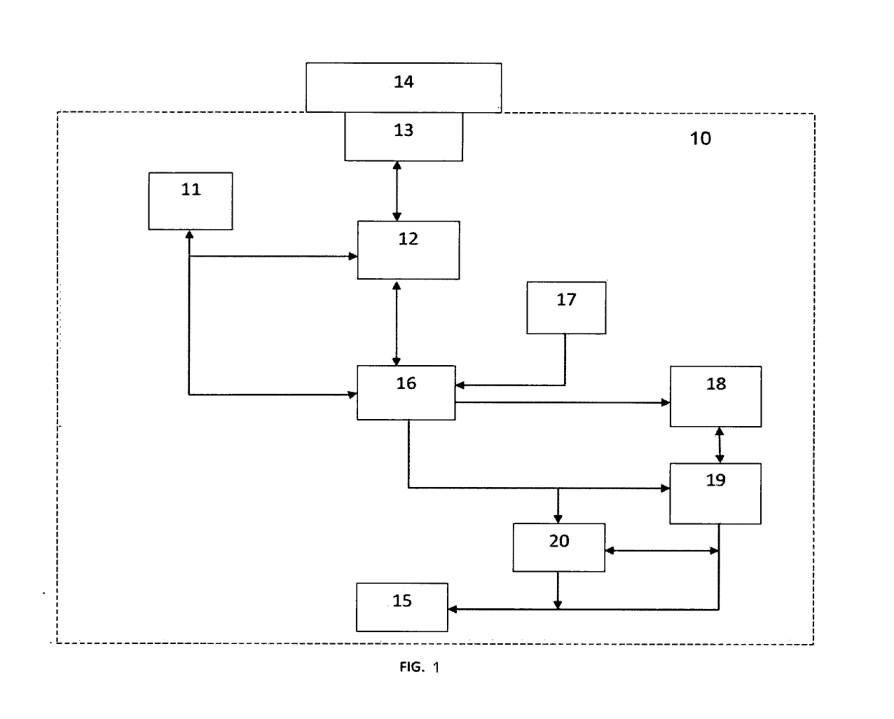

FIG. 1 illustrates an apparatus of the object of this invention which can perform simultaneous application of two different visualization methods: B-scan (two-dimensional) image and two-frequency attenuation method image by applying the same B-scan transducer.

The apparatus 10 comprises a system for visualization of the tissue structure images and upon indication of abnormal tissue images to apply a two-frequency method for determination of the type of abnormalities or state of the tissue being under examination by obtaining an objective attenuation data which allows to differentiate the type of the tissue.

The manner in which the apparatus 10 is used, as will be described in more detail hereinafter, is determined by the arbitrary waveform pulse generator and multi-element transducer used to perform a new combined diagnostic examination.

The apparatus 10 as shown in FIG. 1 includes means 11 an arbitrary waveform generator for generating pulses at desired two different frequencies with a desired amplitude, width and shape. The apparatus 10 further includes means 12 scanner system with means 13 a multi-element transducer which transmits and receives signals on two different frequencies with certain width and shape from arbitrary waveform generator 11. These characteristics of the generator pulses requires for application of the two-frequency attenuation method where each piezo-element must work independently and must receive strong enough echo-signals from desired depth of the tissue being under examination. Operator can place means 13 multi-elements transducer on the organ of the patient 14 being under investigation and display B-scan image for visualization on means 15 monitor by using means 16 computer system with a program memory and known on the market control systems with all kind of features necessary for examination and analyses of the B-scan images.

When on the screen of the B-scan image will appear abnormal structural image of the tissue matter under investigation Operator must find an attenuation information from that spot of interest on two-frequency method image. For that Operator must bring on the screen manually or by using means 17 voice recognition device the indicator line (white line). The indicator line will show from what direction reflected echo-signals are coming and in the beginning white line starts from the piezo-element which is placed in a center of the transducer. Operator can choose any spot of interest on that image and move the white line to chosen spot of interest by pushing manually the buttons corresponding to the desired direction or by using means 17 voice recognition device to say the number of the button or words “left”, “right”, “up” and “down”.

Operator can choose also an area of interest from the two-dimensional structural image of organ or tissue being under examination. Operator can visually analyze on the same screen the reflected signals on two different frequencies to be sure that echo-signals contain objective acoustic information. If there is doubt about it operator can repeat taking echo-signals from that spot of interest until goal is achieved. Operator can use a reflected signal memory 18 and means 19 display memory which give possibilities to Operator to bring back on the screen the previous images on both methods and to go over the obtained information. There is an interference phenomena during the propagation of the ultrasound waves in the media like tissue structure which influences on amplitude of the reflected signals differently on different frequencies. It is possible to avoid this influence on result of the measurement of the attenuation coefficient by analyzing the reflected echo-signals. Operator should try to register the reflected echo-signals on both frequencies at about the same time with about the same shape and width and the interference phenomena can be brought to a neglectable level. The apparatus 10 offers two type of the echo-signal analyzes: manual—visual on screen and automatic—by means 20 two-frequency A-mode analyzer.

It is the object of this invention to make existing on the market any type of ultrasound B-scan imaging apparatus be able to work together with two-frequency attenuation imaging apparatus to improve the quality of the diagnostic examination to determine the type of tissue or the state of tissue being under investigation by applying the physical bodies of these two different transducers separately. Operator can start examination of the patient with a B-scan transducer and when on the screen will appear abnormal image of the tissue structure Operator must put aside the B-scan transducer and apply the two-frequency A-mode transducer on that area of abnormal image.

It is another object of this invention to apply together the bodies of these two different type of transducers as attached to each other that is the body of an A-mode transducer is attached for instance, in the middle of the lateral side of B-scan transducer. The examination of the patient can be started with B-scan part of that combined transducer and after having on the screen an abnormal image of the tissue structure Operator can slide the body of that combined transducer to bring the A-mode part of it to that spot of interest.

It is still another subject of this invention to apply these two methods together by permanently inserting a single piezo-element into the multi-element B-scan transducer. This single piezo-element will be a transducer for the two-frequency attenuation method which will transmit and receive pulses for two-frequency method. The single piezo-element can be placed, for instance, in the middle of the multi-element B-scan transducer. During the examination of the patient Operator can bring the spot of interest to the middle of the displayed on the screen two-dimensional image and to use the two-frequency A-mode transducer to get two-frequency attenuation image displayed on another part of the screen.

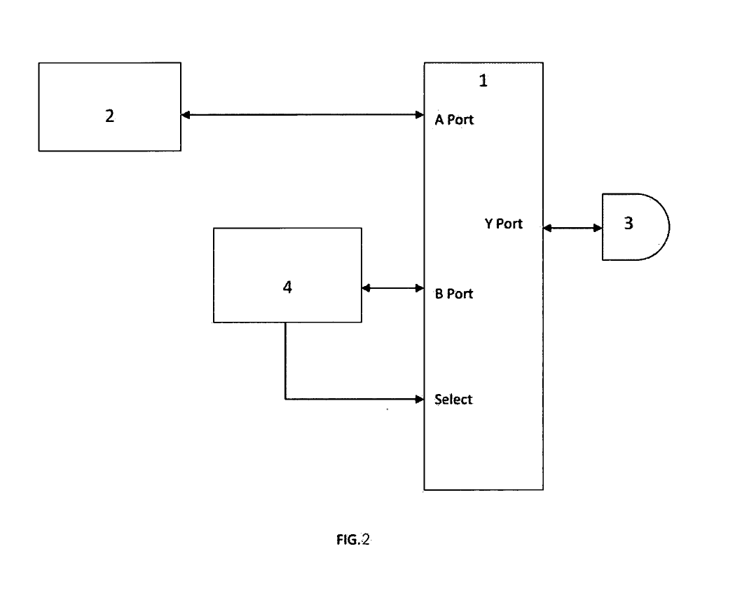

It is still further object of this invention to apply together a two-dimensional (B-scan) visualization method and a two-frequency attenuation method image by using one B-scan transducer only. FIG. 2 illustrates a block diagram of an adapter to the existing on the market any B-scan diagnostic apparatus with multi-elements transducer to switch some of its piezo-elements from B-scan image mode to the Two-Frequency Method image mode.

The adapter works through Analog Switch 1 which is a TTL logic level signal. When it is driven with a logic “1”, the ultrasound machine's system 2 is connected to the transducer head 3. Also, since it is “pulled up”, when nothing is connected to the input, the ultrasound machine's system 2 is connected to the transducer head 3. When it is driven with a logic “0” (or shorted to ground) all elements are disconnected from the ultrasound machine's system 2 and the “IN” signal from Two-Frequency Apparatus 4 through B-port of the Analog Switch 1 is connected to the central elements of the transducer head 3. The adapter-switch consist of two types of connectors which depends on applied ultrasound diagnostic machine and chosen B-scan transducer for that machine. The front face of the adapter which connected with ultrasound machine has the same type of connector as B-scan transducer has and on the lateral side of the adapter where the B-scan transducer connected there is the same type of connector as diagnostic machine has for B-scan transducer connection. Inside of the box of the adapter there is a PC board with relays and all piezo-elements connections in and out of the relays with a power supply wires. Inside in the center of along side of the adapter stands a long rod with a handle to lock and unlock the adapter from the ultrasound machine's connector.

The mentioned above existing on the market B-scan transducers consists of multiple piezo-elements which transmit and receive ultrasound pulses to create a two-dimensional image of the tissue being under investigation. Depend on the manufacturing companies and the purpose of the application of the B-scan transducers the number of the piezo-elements could reach 256 elements. Two-frequency method require a one only transmitter-receiver source and also the excitation of the piezo-elements for two-frequency method is different than for B-scan image. For two-frequency method should be applied generator with variable frequencies, width and shape of the excitation pulses. Depend on depth of the tissue being under investigation the number of the piezo-elements applied as a one transmitter-receiver source will be different. For instance, for B-scan transducers with 128 piezo-elements the number of elements as one transmitter-receiver source can be started from group of 16 piezo-elements situated in the middle of the transducer. If it is not enough strong pulses for chosen depth then can be added second group of 8 piezo-elements to the first one. If these two groups of 24 piezo-elements as one transmitter-receiver source still is not enough than could be added extra 8 piezo-elements to make it 32 piezo-elements as one transmitter-receiver source. For the relay can be used high voltage switch IC which provides switching of the center 32 elements between two-frequency attenuation apparatus and ultrasound B-scan apparatus. The remaining 96 elements have a single switch for each element so that all 128 elements see the same impedance when driven by the ultrasound diagnostic machine. These switches are opened disconnecting the diagnostic apparatus's system from the transducer when the external transceiver is connected to the center 32 elements.

It is apparent that there has been provided in accordance with this invention an apparatus for non-invasively determining a type of tissue matter and/or its state within a living entity which fully satisfies the objects, means and advantages set forth hereinbefore. While the invention has been described in combination with specific embodiments thereof, it is evident that many alternatives, modifications, and variations will be apparent to those scilled in the art in light of the foregoing description. Accordingly, it intended to embrace all such alternatives, modifications, and variations as fall within the spirit and broad scope of the appended claims.

1-6. (canceled)

7. An apparatus for non-invasively determining at least one of a type of a tissue matter and a state of said tissue matter in a living body, said apparatus comprising:
a scanner system including a B-scan imaging transducer to be placed on said tissue matter of said living body;
a waveform generator for generating pulses at two different frequencies and supplying said pulses to said B-scan imaging transducer;
a monitor for displaying two B-scan images generated by said B-scan imaging transducer; and
a two-frequency A-mode transducer for examining said tissue matter to determine said at least one of a type of said tissue matter and a state of said tissue matter.

8. The apparatus of claim 7, wherein said A-mode transducer is attached to said B-scan imaging transducer.

9. The apparatus of claim 8, wherein said A-mode transducer is attached in a middle of a lateral side of said B-scan imaging transducer.

10. The apparatus of claim 7, wherein said A-mode transducer comprises a piezo-element inserted into said B-scan imaging transducer.

11. The apparatus of claim 7, wherein said B-scan imaging transducer has a plurality of piezo-elements and said A-mode transducer comprises some of said piezo-elements which have been switched to a two frequency A-mode.

12. The apparatus of claim 11, further comprising an adapter for switching said some of said piezo-elements to said two frequency A-mode.

13. A method for non-invasively determining at least one of a type of a tissue matter and a state of said tissue matter in a living body, said method comprising the steps of:
providing a scanner system including a B-scan imaging transducer to be placed on said tissue matter and a waveform generator for generating pulses at two different frequencies;
supplying said pulses at two different frequencies to said B-scan imaging transducer and generating two-dimensional B-scan images;
displaying said two-dimensional B-scan images;
choosing a region of interest on said two-dimensional B-scan images;
creating a two-frequency attenuation A-mode image of said region of interest using a two-frequency A-mode transducer; and
analyzing said two-frequency attenuation A-mode image to determine said at least one of said type of tissue matter and said state of said tissue matter.

14. The method of claim 13, wherein said step of choosing a region of interest comprises using push buttons corresponding to a desired direction.

15. The method of claim 13, wherein said step of choosing a region of interest comprises applying a voice recognition device.

16. The method of claim 13, wherein said displaying step comprises displaying said two-dimensional B-scan images on a monitor.

17. The method of claim 16, wherein said choosing step comprises creating an indicator line on said monitor where said two dimensional B-scan images are being displayed to show a direction from which reflected echo signals are coming and moving said indicator line to said region of interest.

18. The method of claim 13, wherein said B-scan imaging transducer and said two frequency A-mode transducer are used sequentially.

19. The method of claim 13, wherein said B-scan imaging transducer and said two frequency A-mode transducer are used simultaneously.

20. The method of claim 13, further comprising attaching said two frequency A-mode transducer to a side of said B-scan imaging transducer.

21. The method of claim 13, further comprising inserting a piezo element into the B-scan imaging transducer and using said piezo element as said two frequency A-mode transducer.
